(19) United States
(12) Patent Application Publication
(10) Pub. No.: US 2005/0053634 A1
(43) Pub. Date: Mar. 10, 2005

(54) Talc-containing cosmetic cleansing emulsions

(75) Inventors: Stephan Ruppert, Hamburg (DE); Michaela Kohut, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/864,482

(22) Filed: Jun. 10, 2004

Related U.S. Application Data
(63) Continuation of application No. PCT/EP02/13908, filed on Dec. 9, 2002.

(30) Foreign Application Priority Data
Dec. 13, 2001 (DE) 101 61 171.4

(51) Int. Cl. A61K 7/06; A61K 7/11

(52) U.S. Cl. 424/401; 424/70.16

(57) ABSTRACT

A cosmetic or dermatological cleansing emulsion comprising one or more detergent surfactants having an HLB value of higher than 15, one or more oil components, talc, one or more polyacrylates and water.

### CROSS-REFERENCE TO RELATED APPLICATIONS

The present application is a continuation of International Application No. PCT/EP02/13908, filed Dec. 9, 2002, the entire disclosure whereof is expressly incorporated by reference herein, which claims priority under 35 U.S.C. § 119 of German Patent Application No. 101 61 171.4, filed Dec. 13, 2001.

### BACKGROUND OF THE INVENTION

1. Field of the Invention

The present invention relates to talc-containing cosmetic cleansing emulsions, in particular those which do not contain an emulsifier in the conventional sense.

2. Discussion of Background Information

The production of cosmetic cleansing compositions has for years shown an increasing tendency. This is to be attributed in particular to the increasing health awareness and hygiene requirement by the consumer.

Cleansing means the removal of (environmental) dirt and thus brings about an increase in psychological and physical wellbeing. The cleansing of the surface of skin and hair is a very complex operation dependent on many parameters. Firstly, external substances, such as, for example, hydrocarbons, or inorganic pigments from very diverse surroundings, as well as residues of cosmetics and also undesired microorganisms are to be removed as completely as possible. Secondly, excretions endogenous to the body, such as perspiration, sebum, skin and hair flakes, are to be washed away without serious interventions in the physiological equilibrium.

The requirements placed on the properties of cosmetic cleansing preparations have changed radically in recent years. Previously, effects such as cleansing and lathering were at the forefront of consumer wishes. Currently, the ecological, economic and in particular dermatological properties of the products are a priority, although the ability to lather continues to play a decisive role, for example as an indicator to remove residual amounts of surfactants after cleansing skin and hair, or to avoid overdoses during application. However, in the case of cosmetic products—in contrast to most technical cleaning compositions—the compatibility with skin and mucosa is definitely at the forefront; the products should be “mild”.

Cosmetic or dermatological cleansing preparations are so-called “rinse off” preparations which are rinsed off the skin following application. They are generally applied to the areas of the body to be cleansed in the form of a foam with water. The basis of all cosmetic or dermatological cleansing preparations is detergentsurfactants. Surfactants are amphiphilic substances which are able to dissolve organic, nonpolar substances in water. They are characterized by ambivalent behavior toward water and lipids: the surfactant molecule contains at least one hydrophilic group and one lipophilic group, which permit positioning at the interface between these two classes of substance. In this way, surfactants reduce the surface tension of water, wet the skin, facilitate soil removal and dissolution, provide for easy rinsing and—if desired—also for foam regulation. This is the basis for the soil removal of lipid-containing soilings.

The hydrophilic moieties of a surfactant molecule are mostly polar functional groups, for example —COO⁻, —OSO₃²⁻, —SO₃⁻, whilst the hydrophobic moieties are usually nonpolar hydrocarbon radicals. Surfactants are generally classified according to the type and charge of the hydrophilic molecular moiety. In this respect, there are four different groups:
anionic surfactants,
cationic surfactants,
amphoteric surfactants and
nonionic surfactants.

Anionic surfactants generally have carboxylate, sulfate or sulfonate groups as functional groups. In aqueous solution in an acidic or neutral medium they form negatively charged organic ions. Cationic surfactants are almost exclusively characterized by the presence of a quaternary ammonium group. In aqueous solution in an acidic or neutral medium they form positively charged organic ions. Amphoteric surfactants contain both anionic and cationic groups and, in aqueous solution, accordingly behave as anionic or cationic surfactants, depending on the pH. In a highly acidic medium, they have a positive charge and in an alkaline medium, a negative charge. In the neutral pH range, however, they are zwitterionic, as the following example illustrates:
RNH₂⁺CH₂CH₂COOH X⁻ (at pH=2) X⁻=any anion, e.g. Cl⁻
RNH₂⁺CH₂CH₂COO⁻ (at pH=7)
RNHCH₂CH₂COO⁻B⁺ (at pH=12) B⁺=any cation, e.g. Na⁺

Polyether chains are typical nonionic surfactants. Nonionic surfactants do not form ions in an aqueous medium.

The detergent surfactants in cosmetic and dermatological cleansing compositions are subject to very critical assessment with regard to their dermatological and ecological behavior. The latter is of importance particularly since they are used in a considerable amount and, in accordance with directions, pass into waste water following use.

Starting from the already described central importance of detergent surfactants for the cleansing operation, their behavior on the human skin is of very great importance.

Even cleansing of the skin with the aid of water—without the addition of surfactants—results initially in swelling of the horny layer of the skin. The degree of this swelling depends, inter alia, on the duration of the bath and its temperature. At the same time, water-soluble substances are washed off or out, such as, for example, water-soluble dirt constituents, but also the skin's own substances, which are responsible for the water-binding capacity of the horny layer. By means of the skin's own surface-active substances, skin fats are moreover also dissolved to a certain extent and washed out. After initial swelling, this causes a subsequent drying out of the skin.

It is understandable that detergent surfactants which are intended to cleanse the skin and hair of greasy and water-soluble dirt constituents also have a defatting action on the normal skin lipids. In any cleansing of the skin, intercorneocytic lipids and sebum constituents are also removed to a varying degree. This means that the natural water/lipid mantle of the skin is more or less destroyed during any washing operation. This can lead, particularly in the case of extreme defatting, to a short-term change in the barrier function of the skin, where, of course, also the particular condition of the area of skin treated is of considerable influence on the changes shown. For example, the skin thickness, the number of sebaceous glands and sweat glands and the sensitivity associated therewith can vary considerably.

In principle, it is accordingly regarded as a requirement of detergent surfactants that they are as biologically inactive as possible in order to avoid undesired side-effects. They should display their cleansing action with optimum mildness, best skin compatibility and low defatting.

In addition, however, there has been no lack of attempts to find suitable cleansing preparations which regenerate or “refat” the skin at the same time with good cleansing power. However, the performance achieved often remains below that expected, meaning that the user generally has to resort to separate care products which are applied to the skin after cleansing and remain on this (so-called “leave-on” products).

These “leave-on” products generally also comprise a number of active ingredients which care for and regenerate the skin. They increase the barrier properties of the skin and decrease and prevent premature skin aging (e.g. wrinkles and lines). It is their object to help the skin to a smooth and healthy youthful appearance.

Another object of the active ingredients is the acceleration of skin regeneration, which leads to a more rapid restoration of the natural equilibrium of the skin after washing with skin-irritating surfactants. A further object of the active ingredients is the replacement of lipids, humectant factors, vitamins and other skin constituents washed out during the washing process. In addition, active ingredients (UV filters) can serve for the protection of the skin from the harmful UV radiation of sunlight.

As a rule, cosmetic or dermatological cleansing preparations are very well tailored to an assumed application spectrum since for a defined, mild cleansing action, the mechanical parameters—such as, for example, the time factor—which are different depending on the application are in particular also of considerable importance. This becomes clear, for example, if the different application (contact) times of a foam bath in comparison to brief hand-washing are considered.

Cosmetic cleansing compositions usually comprise mixtures of surfactants of various types. The choice is orientated primarily to the skin compatibility and the desired cosmetic performance of the surfactants. In addition, foaming power, ability to be formulated and a favorable performance/cost ratio play an important role.

Liquid soaps or washing lotions are not only used for cleansing the hands, but generally also for the whole body, including the face. They are accordingly also suitable for use as a shower preparation. In the development of these products, the dermatological requirements are foremost, since the skin comes into intensive contact with the concentrated surfactant solution. Particular value is therefore placed on the choice of mild surfactants in low concentration. Further criteria are furthermore a good foaming power and a pleasant, refreshing fragrance and the simultaneous care of the skin. Washing lotions and in particular shower baths generally have viscosities of from about 3000 to 10 000 mPa.s, which on the one hand allow good dispersibility of the product with rapid foaming, but on the other hand should be high enough in order to allow flawless application by hand or flannel.

Liquid soaps or washing lotions are generally characterized by a greater or lesser water content, but as a rule develop no noteworthy care effect since they only have a low oil content.

A relatively new technical development concerns surfactant-containing shower preparations with a high oil content. German laid-open specification 44 24 210 in this connection describes cosmetic or dermatological shower preparations with a surfactant content of at most 55% by weight and an oil content of more than 45% by weight, the preparations being essentially water-free. Owing to the high oil content, these preparations have a regenerating effect with respect to the general condition of the skin. In this case, they at the same time have good foam development and high cleansing power.

In addition, WO 96/17591 describes foaming liquid skin cleansing compositions which comprise the following substances: 5 to 30% by weight of a moisture-donating active ingredient, which has a Vaughan solubility parameter (VSP) of 5 to 10, 0.3 to 5% by weight of a water-dispersible gel-forming polymer, 5 to 30% by weight of a synthetic surface-active substance, 0 to 15% by weight of a C₈ to C₁₄ fatty acid soap and water, where the preparations have a lipid deposition value (LDV) of at least 5 to 1000 and in which the synthetic surface-active substance and the soap have a common CMC equilibrium surface tension value of 15 to 50. However, this specification was unable to point the way to the present invention.

Conventional cleansing preparations with a high oil content generally have the disadvantage of leaving behind a slightly dull, sticky feel on the skin after washing. However, in most cases users prefer a silky-smooth feel on the skin. This is usually brought about by adding so-called “polyquats”, i.e. quaternary ammonium salts or ethoxylated oils to the preparations. However, since these compounds have only low adhesion (substantivity) on the skin, this effect during the application of cleansing preparations is marginal.

For the cleansing and simultaneous care of the skin, the prior art also recognizes emulsion-based cleansing products. These are formulated by stabilizing the emulsion with emulsifiers and subsequently tailoring a surfactant system.

Emulsifiers also have an amphiphilic structure, and are thus comparable to the surfactants as far as the structure is concerned. Emulsifiers allow or facilitate the uniform distribution of two or more mutually immiscible phases and at the same time prevent their separation. Since emulsions are in general destroyed by the addition of surfactants, the choice of surfactant system is severely restricted, and the cleansing preparations obtained are based on expensive and complicated formulations.

What then distinguishes detergent surfactants from emulsifiers?

At the end of the 1940s a system was developed which was intended to facilitate the choice of emulsifiers. Each emulsifier is given a so-called HLB value (a dimensionless number between 0 and 20) which indicates whether a preferred water-solubility or oil-solubility is present. Numbers below 9 indicate oil-soluble, hydrophobic emulsifiers, numbers above 11 water-soluble, hydrophilic emulsifiers. The HLB value says something about the equilibrium of the size and strength of the hydrophilic and the lipophilic groups of an emulsifier. It can be derived from these considerations that also the effectiveness of an emulsifier can be characterized by its HLB value. The following list shows the relationship between HLB value and possible field of application:

| HLB value | Field of application |
|---|---|
| 0 to 3 | Antifoam |
| 3 to 8 | W/O emulsifier |
| 7 to 9 | Wetting agent |
| 8 to 18 | O/W emulsifier |
| 12 to 18 | Solubility promoter |

The HLB value of an emulsifier can also be composed of increments, where the HLB increments for the various hydrophilic and hydrophobic groups from which a molecule is composed can be found in tables. In this way, HLB values can in principle also be determined for detergent surfactants, although the HLB system was originally only conceived for emulsifiers. It is found that detergent substances generally have HLB values which are significantly greater than 20.

It would be desirable to have available cleansing preparations based on emulsions which remedy the disadvantages of the prior art and which are based on simple, cost-effective formulations. The preparations should have a high care action without the cleansing action taking second place to it. Furthermore, it would be desirable to formulate the cleansing preparations such that a silky-smooth feel on the skin is left after application by the consumer.

The present invention further relates to detergent hair cosmetic preparations, generally referred to as shampoos. In particular, the present invention relates to hair cosmetic active ingredient combinations and preparations for the care of hair and of the scalp. The prior art lacks shampoo formulations which are able to bestow care on damaged hair in a satisfactory manner. An object was therefore also to remedy these disadvantages of the prior art.

### SUMMARY OF THE INVENTION

The present invention provides a cosmetic or dermatological cleansing emulsion comprising (a) from 1% to 30% by weight of one or more detergent surfactants which have an HLB value of higher than 15; (b) from 35% to 50% by weight of one or more oil components; (c) from 0.1% to 10% by weight of talc; (d) from 0.2% to 5% by weight of one or more polyacrylates selected from anionic homopolymers and/or anionic copolymers of acrylic acid and/or alkylated acrylic acid(s) and/or esters thereof; and (e) from 5% to 60% by weight of water.

In one aspect of the emulsion, the one or more detergent surfactants may have an HLB value of higher than 25, e.g., an HLB value of higher than 35.

In another aspect, the one or more detergent surfactants may comprise one or more anionic surfactants, for example, an acylamino acid and/or a salt thereof, a sulfonic acid and/or a salt thereof, and/or a sulfuric acid ester. Preferably, the one or more anionic surfactants comprise at least sodium laureth sulfate.

In yet another aspect, the one or more detergent surfactants may comprise one or more cationic surfactants, for example, at least one quaternary surfactant.

In a still further aspect, the one or more detergent surfactants may comprise one or more amphoteric surfactants, e.g., an acyl-/dialkylethylenediamine.

In another aspect, the one or more detergent surfactants may comprise one or more nonionic surfactants, e.g., an alkanolamide, an ester formed by esterification of a carboxylic acid with at least one of ethylene oxide, glycerol and sorbitan, and/or an ether.

In yet another aspect, the emulsion may comprise from 5% to 25% by weight of component (a), e.g., from 10% to 20% by weight of component (a).

In another aspect of the emulsion of the present invention, component (a) may comprise at least one of sodium acyl glutamate, myristoyl sarcosine, TEA lauroyl sarcosinate, sodium lauroyl sarcosinate and sodium cocoyl sarcosinate, sodium/ammonium cocoyl isethionate, dioctyl sodium sulfosuccinate, disodium laureth sulfosuccinate, disodium lauryl sulfosuccinate and disodium undecyleneamido MEA sulfosuccinate, sodium, ammonium, magnesium, MIPA, TIPA laureth sulfate, sodium myreth sulfate and sodium C₁₂₋₁₃ pareth sulfate, sodium, ammonium and TEA lauryl sulfate, benzalkonium chloride, alkylbetaine, alkylamidopropylbetaine and alkylamidopropylhydroxysultaine, sodium acyl-amphoacetate, disodium acylamphodipropionate, disodium alkylamphodiacetate, sodium acylamphohydroxypropylsulfonate, disodium acylamphodiacetate and sodium acylamphopropionate, cocamide MEA/DEA/MIPA, lauryl glucoside, decyl glycoside and coconut glycoside.

In a still further aspect of the emulsion, component (b) may comprise one or more of 2-ethylhexyl iso-stearate, octyldodecanol, isotridecyl isononanoate, isoeicosane, 2-ethylhexyl cocoate, C₁₂₋₁₅ alkyl benzoate, caprylic/capric acid triglyceride, and dicaprylyl ether, for example, a mixture of C₁₂₋₁₅ alkyl benzoate and 2-ethyl hexyl isostearate, a mixture of C₁₂₋₁₅ alkyl benzoate and isotridecyl isononanoate, or a mixture of C₁₂₋₁₅ alkyl benzoate, 2-ethyl-hexyl isostearate and isotridecyl isononanoate.

In another aspect of the emulsion of the present invention, component (c) may comprise talc having an average particle size of from 1 μm to 20 μm.

In yet another aspect, the emulsion may comprise from 0.5% to 5% by weight of component (c), and/or up to 3% by weight of component (c).

In another aspect of the emulsion, component (d) may comprise a polymer of at least one of acrylic acid and esters thereof, and/or acrylate/alkyl acrylate copolymers. For example, the emulsion of the present invention may comprise at least one anionic surfactant and at least one C₁₀-C₃₀ alkyl acrylate polymer.

In a still further aspect, the emulsion of the present invention may comprise from 0.5% to 2% by weight of component (d).

The present invention also provides a cosmetic or dermatological cleansing emulsion which comprises from 5% to 20% by weight of component (a); from 35% to 50% by weight of component (b); from 0.5% to 5% by weight of component (c); from 0.5% to 2% by weight of component (d); and from 5% to 55% by weight of component (e).

Components (a) to (d) include the various aspects thereof, as recited above.

The present invention further provides several products which comprise the emulsion of the present invention, including the various aspects thereof, e.g., a foam bath, a shower bath, a tub bath, a hair shampoo, and a cream-like preparation.

The present invention also provides a method for the treatment or prophylaxis of inflammatory skin conditions, and a method of protecting sensitive and dry skin. These methods comprise the application of the emulsion of the present invention, including the various aspects thereof, onto at least parts of the skin.

It has surprisingly been found,that cosmetic or dermatological cleansing emulsions comprising, based on the total weight of the preparations:
1 to 30% by weight of one or more detergent surfactants selected from surfactants which have an HLB value of more than 15,
35 to 50% by weight of one or more oil components,
0.1 to 10% by weight of talc,
0.2 to 5% by weight of one or more polyacrylates selected from anionic homopolymers and/or copolymers of acrylic acid and/or alkylated acrylic acid derivatives, and esters thereof and
5 to 60% by weight of water,
   overcome the disadvantages of the prior art.

In this connection, talc particles with an average particle size of from 1 to 20 μm and talc amounts of up to 3% by weight based on the total weight of the preparation, are particularly preferred according to the invention.

Talc is a widespread hydrated magnesium silicate of the approximate composition Mg₃[(OH)₂/Si₄O₁₀] or 3MgO.4SiO₂, whose denser aggregates are called steatite. Talc forms transparent to nontransparent, predominantly colorless, white or pale green, completely cleavable masses, which consist of laminocrystalline, flaky, mica-like aggregates with a mother-of-pearl luster. Natural talc usually consists of traces of other metal oxides. The following composition is thus specified for typical talc: 61% SiO₂, 31% MgO, 5% H₂O, 1.4% Al₂O₃, 1.1% FeO, 0.3% CaO, 0.1% COO₂. Further trace elements which may be present are Mn, Ti, Cr, Ni, Na and K; OH can be partially replaced by F.

Although DE 197 14 829 also describes cosmetic cleansing compositions which may also comprise talc, this specification was unable to point the way to the present invention since the cleansing compositions disclosed therein do not comprise polyacrylates.

The cosmetic and/or dermatological cleansing emulsions for the purposes of the present invention are based on simple and cost-effective formulations. At the same time, they have good foam development and high cleansing power. Owing to the high oil content, these preparations have a regenerating action with respect to the general condition of the skin, reduce the feeling of skin dryness and make the skin supple and silky-smooth.

The cleansing emulsions advantageously comprise one or more washing-active anionic, cationic, amphoteric and/or nonionic surfactants according to the invention. It is particularly advantageous to choose the detergent surfactants according to the invention from surfactants which have an HLB value of more than 25, those which have an HLB value of more than 35 being very particularly advantageous.

Particularly advantageous detergent anionic surfactants for the purposes of the present invention are
acylamino acids and salts thereof, such as
   acylglutamates, in particular sodium acylglutamate
   sarcosinates, for example myristoyl sarcosine, TEA lauroyl sarcosinate, sodium lauroyl sarcosinate and sodium cocoyl sarcosinate,
sulfonic acids and salts thereof, such as
   acyl isethionates, e.g. sodium/ammonium cocoyl isethionate,
   sulfosuccinates, for example dioctyl sodium sulfosuccinate, disodium laureth sulfosuccinate, disodium lauryl sulfosuccinate and disodium undecylenamido MEA sulfosuccinate
and sulfuric esters, such as
   alkyl ether sulfate, for example sodium, ammonium, magnesium, MIPA, TIPA laureth sulfate, sodium myreth sulfate and sodium C₁₂₋₁₃ pareth sulfate,
   alkyl sulfates, for example sodium, ammonium and TEA lauryl sulfate.

Particularly advantageous detergent cationic surfactants for the purposes of the present invention are quaternary surfactants. Quaternary surfactants contain at least one N atom which is covalently bonded to four alkyl or aryl groups. Benzalkonium chloride, alkylbetaine, alkylamidopropylbetaine and alkylamidopropylhydroxysultaine are advantageous.

Particularly advantageous detergent amphoteric surfactants for the purposes of the present invention are
acyl-/dialkylethylenediamines, for example sodium acyl amphoacetate, disodium acyl amphodipropionate, disodium alkylamphodiacetate, sodium acyl amphohydroxypropylsulfonate, disodium acyl amphodiacetate and sodium acyl amphopropionate.

Particularly advantageous detergent nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers and alkyl polyglycosides, such as lauryl glycoside, decyl glycoside and cocoglycoside.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate,
phosphoric esters and salts, such as, for example, DEA-oleth-10 phosphate and dilaureth-4 phosphate,
alkylsulfonates, for example sodium cocosmonoglyceride sulfate, sodium C₁₂₋₁₄₋ olefinsulfonate, sodium lauryl sulfoacetate and magnesium PEG-3 cocamidesulfate.

Further advantageous amphoteric surfactants are
N-alkylamino acids, for example aminopropylalkylglutamide, alkylaminopropionic acid, sodium alkylimidodipropionate and lauroamphocarboxyglycinate.

Further advantageous nonionic surfactants are alcohols.

Further suitable anionic surfactants for the purposes of the present invention are also
acyl glutamates, such as di-TEA-palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen
and carboxylic acids and derivatives, such as
   for example lauric acid, aluminum stearate, magnesium alkanolate and zinc undecylenate,
   ester carboxylic acids, for example calcium stearoyl lactylate, laureth-6 citrate and sodium PEG-4 lauramide carboxylate,
   alkylarylsulfonates.

Further suitable cationic surfactants for the purposes of the present invention are also
alkylamines,
alkylimidazoles and
ethoxylated amines.

Further suitable nonionic surfactants for the purposes of the present invention are also amine oxides, such as cocoamidopropylamine oxide.

It is advantageous for the purposes of the present invention when the content of one or more detergent surfactants in the cosmetic or dermatological cleansing emulsion is chosen from the range from 5 to 25% by weight, very particularly advantageously from 10 to 20% by weight, in each case based on the total weight of the preparations.

The oil phase of the cosmetic or dermatological cleansing emulsions for the purposes of the present invention is advantageously chosen from esters of saturated and/or unsaturated, branched and/or unbranched alkane carboxylic acids with a chain length of from 3 to 30 carbon atoms and saturated and/or unsaturated, branched and/or unbranched alcohols with a chain length of from 3 to 30 carbon atoms, from esters of aromatic carboxylic acids and saturated and/or unsaturated, branched and/or unbranched alcohols with a chain length of from 3 to 30 carbon atoms. Such ester oils can then advantageously be chosen from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from branched and unbranched hydrocarbons and hydrocarbon waxes, silicone oils, dialkyl ethers, saturated or unsaturated, branched or unbranched alcohols, and fatty acid triglycerides, namely triglycerol esters of saturated and/or unsaturated, branched and/or unbranched alkane carboxylic acids with a chain length of from 8 to 24, in particular 12 to 18 carbon atoms. The fatty acid trigycerides can, for example, advantageously be selected from synthetic, semi-synthetic and natural oils, e.g. olive oil, sunflower oil, soybean oil, peanut oil, rapeseed oil, almond oil, palm oil, coconut oil, palm kernel oil and the like.

Any desired mixtures of such oil and wax components may also be used advantageously for the purposes of the present invention. In some instances, it may also be advantageous to use waxes, for example cetyl palmitate, as the sole lipid component of the oil phase.

The oil phase is advantageously chosen from 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

Advantageously, the oil phase can also have a content of cyclic or linear silicone oils or consist entirely of such oils, where, however, it is preferred to use an additional content of other oil phase components apart from the silicone oil or the silicone oils.

Advantageously, cyclomethicone (octamethylcyclotetrasiloxane) is used as the silicone oil to be used according to the invention. However, other silicone oils may also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The oil phase is also advantageously chosen from phospholipids. The phospholipids are phosphoric esters of acylated glycerols. Of very great importance among the phosphatidyl cholines are, for example, the lecithins, which are characterized by the general structure 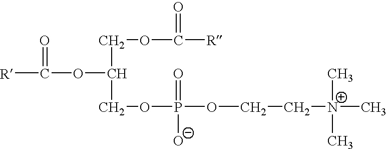
where R′ and R″ are typically unbranched aliphatic radicals having 15 or 17 carbon atoms and up to 4 cis double bonds.

Advantageous polyacrylates according to the invention are polymers of acrylic acid, in particular those which are chosen from the so-called carbomers or carbopols (Carbopol® is actually a registered trademark of the B.F. Goodrich company). Polyacrylates are compounds of the general structural formula 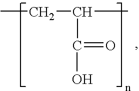
whose molecular weight can be between about 400,000 and more than 4,000,000. The group of polyacrylates also includes acrylate/alkyl acrylate copolymers, for example those which are characterized by the following structure:

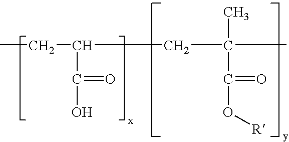

In the above formula, R′ is a long-chain alkyl radical and x and y are numbers which symbolize the particular stoichiometric proportion of the respective comonomers. These polyacrylates are also advantageous for the purposes of the present invention.

Advantageous carbopols are, for example, the grades 907, 910, 934, 940, 941, 951, 954, 980, 981, 1342, 1382, 2984 and 5984 or alternatively the grades ETD (Easy-to-disperse) 2001, 2020, 2050, where these compounds can be present individually or in any combinations with one another.

Particular preference is given to Carbopol 981, 1382 and ETD 2020 (both individually and also in combination).

Also advantageous for the purposes of the present invention are the copolymers of C₁₀₋₃₀-alkyl acrylates and one or more monomers of acrylic acid, methacrylic acid or esters thereof comparable to the acrylatelalkyl acrylate copolymers. The INCI name of such compounds is “Acrylates/C 10-30 Alkyl Acrylate Crosspolymer”. Those obtainable under the trade names Pemulen TR1 and Pemulen TR2 from the B.F. Goodrich company are particularly advantageous.

It is advantageous for the purposes of the present invention when the content of one or more polyacrylates in the cosmetic or dermatological cleansing emulsion is chosen from the range from 0.5 to 2% by weight, very particularly advantageously from 0.7 to 1.5% by weight, in each case based on the total weight of the preparations.

Into the cleansing emulsions according to the invention it is advantageously possible to incorporate active ingredients which, inter alia, serve for the prophylaxis and/or treatment of inflammatory skin conditions and/or for skin protection in cases of sensitively determined and dry skin (such as, for example, atopic eczema, seborrheic eczema, polymorphous photodermatosis, psoriasis, vitiligo, wound healing disorders, itching, sensitive or irritated skin, photoinduced skin damage and UV-induced immunosuppression, changes in desquamation, changes in normal fibroblast and keratinocyte proliferation, changes in normal fibroblast and keratinocyte differentiation of deficient sensitive or hypoactive skin conditions or deficient sensitive or hypoactive conditions of skin appendages and for reducing skin thickness).

An embodiment of the invention which is particularly preferred according to the invention are cleansing emulsions which comprise, as detergent surfactants, at least one anionic surfactant and at least one thickener based on C₁₀-C₃₀-alkyl acrylates as polyacrylate. Sodium laureth sulfate is in this case particularly preferred as anionic surfactant. This combination of ingredients is characterized by its stability, its foam formation behavior and by its particularly pleasant feel on the skin.

Cosmetic preparations which are cosmetic cleansing preparations for the skin may be in liquid or solid form.

Apart from the abovementioned substances, the compositions optionally comprise, in accordance with the invention, the additives customary in cosmetics, for example perfume, dyes, antimicrobial substances, refatting agents, complexing and sequestering agents, pearlizing agents, plant extracts, vitamins, active ingredients, preservatives, bactericides, pigments which have a coloring action, thickeners, softening, moisturizing and/or humectant substances, or other customary constituents of a cosmetic or dermatological formulation, such as alcohols, polyols, polymers, foam stabilizers, electrolytes, organic solvents or silicone derivatives.

An additional content of antioxidants is generally preferred. According to the invention, favorable antioxidants which can be used are all antioxidants which are suitable or customary for cosmetic and/or dermatological applications.

Advantageously, the antioxidants are selected from amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, lipoic acid and derivatives thereof (e.g. dihydrolipoic acid), aurothioglucose, propylthiouracil and other thiols, (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin, rutinic acid and derivatives thereof, α-glycosylrutin, ferulic acid, furfurylidene glucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these said active ingredients.

The amount of the abovementioned antioxidants (one or more compounds) in the emulsions is preferably 0.001 to 30% by weight, particularly preferably 0.05 to 20% by weight, in particular 0.1 to 10% by weight, based on the total weight of the preparation.

If vitamin E and/or derivatives thereof are the antioxidant of the antioxidants, it is advantageous to choose their respective concentrations from the range from 0.001 to 10% by weight, based on the total weight of the formulation.

If the vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

Surprisingly, it has been found that very diverse active ingredients having different solubilities can be incorporated homogeneously into the cleansing emulsions according to the invention. The substantivity of the active ingredients on skin and hair is significantly higher from the cleansing emulsion described than from conventional surfactant-containing cleansing formulations. It is to be suspected that the washing out of the active ingredients from the skin by the surfactants present in the formula is lessened or at least reduced by the formation of an oil film on the skin, such that a relatively large amount of the active ingredients present in the product remains on the skin.

According to the invention, the active ingredients (one or more compounds) can very advantageously be chosen from the following group:
acetylsalicylic acid, atropine, azulene, hydrocortisone and derivatives thereof, e.g. hydrocortisone 17-valerate, vitamins of the B and D series, very favorably vitamin B₁, vitamin B₁₂, vitamin D₁, vitamin A or derivatives thereof, such as retinyl palmitate, vitamin E or derivatives thereof, such as, for example, tocopheryl acetate, vitamin C and derivatives thereof, such as, for example, ascorbyl glucoside, but also bisabolol, unsaturated fatty acids, namely the essential fatty acids (often also called vitamin F), in particular γ-linolenic acid, oleic acid, eicosapentanoic acid, docosahexanoic acid and derivatives thereof, chloramphenicol, caffeine, prostaglandins, thymol, camphor, squalene, extracts or other products of vegetable and animal origin, e.g. evening primrose oil, borage oil or currant seed oil, fish oils, cod liver oil, but also ceramides and ceramide-like compounds, incense extract, green tea extract, water lily extract, licorice extract, Hamamelis.

It is also advantageous to choose the active ingredients from refatting substances, for example purcellin oil, Eucerit® and Neocerit®.

Particularly advantageously, the active ingredient or the active ingredients are also selected from the NO synthase inhibitors, particularly when the preparations according to the invention are intended to be used for the treatment and prophylaxis of the symptoms of intrinsic and/or extrinsic skin aging, and for the treatment and prophylaxis of the harmful effects of ultraviolet radiation on the skin.

A preferred NO synthase inhibitor is nitroarginine.

The active ingredient or active ingredients are further advantageously selected from catechols and bile esters of catechols and aqueous or organic extracts of plants or parts of plants which have a content of catechols or bile esters of catechols, such as, for example, the leaves of the plant family Theaceae, in particular of the species Camellia sinensis (green tea). Their typical ingredients (such as, for example, polyphenols or catechols, caffeine, vitamins, sugars, minerals, amino acids, lipids) are particularly advantageous.

Catechols are a group of compounds which are to be regarded as being hydrogenated flavones or anthocyanidins and represent derivatives of “catechol” (3,3′,4′,5,7-flavanpentaol, 2-(3,4-dihydroxyphenyl)chroman-3,5,7-triol). Epicatechol ((2R,3R)-3,3′,4′,5,7-flavanpentaol) is also an advantageous active ingredient for the purposes of the present invention.

Also advantageous are plant extracts with a content of catechols, in particular extracts of green tea, such as, for example, extracts of leaves of the plants of the species Camellia spec., very particularly the tea strains Camellia sinenis, C. assamica, C. taliensis or C. irrawadiensis and crossings of these with, for example, Camellia japonica.

Preferred active ingredients are also polyphenols or catechols from the group of (−)-catechol, (+)-catechol, (−)-catechol gallate, (−)-gallocatechol gallate, (+)-epicatechol, (−)-epicatechol, (−)-epicatechol gallate, (−)-epigallocatechol, (−)-epigallocatechol gallate.

Flavone and its derivatives (often also collectively called “flavones”) are also advantageous active ingredients for the purposes of the present invention. They are characterized by the following basic structure (substitution positions indicated):

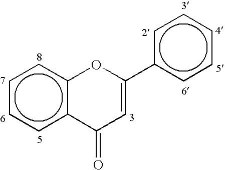

Some of the more important flavones, which can also preferably be employed in preparations according to the invention, are listed in Table 2 below:

TABLE 2

OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaeampferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones usually occur in glycosylated form.

According to the invention, the flavonoids are preferably chosen selected from substances of the generic structural formula 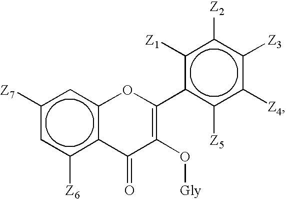
where Z₁ to Z₇, independently of one another, are selected from H, OH, alkoxy and hydroxyalkoxy groups, where the alkoxy or hydroxyalkoxy groups can be branched or unbranched and can have 1 to 18 carbon atoms, and where Gly is selected from mono- and oligoglycoside radicals.

According to the invention, the flavonoids can also advantageously be chosen from the group of substances of the generic structural formula 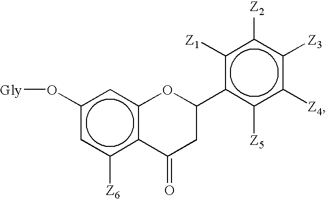
where Z₁ to Z₆, independently of one another, are selected from H, OH, alkoxy and hydroxyalkoxy groups, where the alkoxy or hydroxyalkoxy groups can be branched or unbranched and can have 1 to 18 carbon atoms, and where Gly is selected from mono- and oligoglycoside radicals.

Preferably such structures can be selected from substances of the generic structural formula 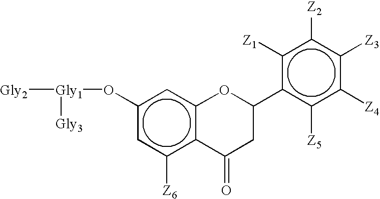
where Gly₁, Gly₂ and Gly₃ independently of one another, are monoglycoside radicals or. Gly₂ and Gly₃ can also individually or together be saturations by hydrogen atoms.

Preferably, Gly₁, Gly₂ and Gly₃, independently of one another, are selected from hexosyl radicals, in particular the rhamnosyl radicals, and glucosyl radicals. However, other hexosyl radicals, for example allosyl, altrosyl, galactosyl, gulosyl, idosyl, mannosyl and talosyl are also in some instances to be used advantageously. It may also be advantageous according to the invention to use pentosyl radicals.

Advantageously, Z₁ to Z₅, independently of one another, are selected from H, OH, methoxy, ethoxy and 2-hydroxyethoxy, and the flavone glycosides have the structure

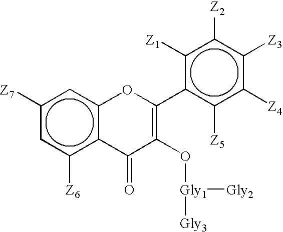

Particularly advantageously, the flavone glycosides according to the invention are from the group which is represented by the following structure: 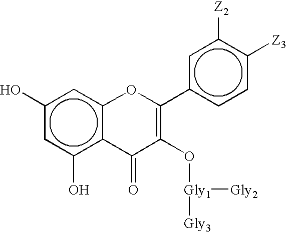
where Gly₁, Gly₂ and Gly₃, independently of one another, are monoglycoside radicals or Gly₂ and Gly₃ can also individually or together be saturations by hydrogen atoms.

Preferably, Gly₁, Gly₂ and Gly₃, independently of one another, are selected from hexosyl radicals, in particular the rhamnosyl radicals and glucosyl radicals. However, other hexosyl radicals, for example allosyl, altrosyl, galactosyl, gulosyl, idosyl, mannosyl and talosyl are also to be used advantageously in some circumstances. It may also be advantageous according to the invention to use pentosyl radicals.

It is particularly advantageous for the purposes of the present invention to select the flavone glycoside or the flavone glycosides from α-glucosylrutin, α-glucosylmyricetin, α-glucosylisoquercitrin, α-glucosylisoquercetin and α-glucosylquercitrin.

According to the invention, α-glucosylrutin is particularly preferred.

Also advantageous according to the invention are naringin (aurantiin, naringenin 7-rhamnoglucoside), hesperidin (3′,5,7-trihydroxy-4′-methoxyflavanone 7-rutinoside, hesperidoside, hesperetin-7-O-rutinoside). Rutin (3,3′,4′,5,7-pentahydroxyflyvone 3-rutinoside, quercetin 3-rutinoside, sophorin, birutan, rutabion, taurutin, phytomelin, melin), troxerutin (3,5-dihydroxy-3′,4′,7-tris(2-hydroxyethoxy)flavone 3-(6-O-(6-deoxy-α-mannopyranosyl)-β-D-glucopyranoside)), monoxerutin (3,3′,4′,5-tetrahydroxy-7-(2-hydroxyethoxy)-flavone 3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxy-flavanone), eriodictyol 7-glucoside (3′,4′,5,7-tetrahydroxyflavanone 7-glucoside), flavanomarein (3′,4′,7,8-tetrahydroxyflavanone 7-glucoside) and isoquercetin (3,3′,4′,5,7-pentahydroxyflavanone-3-(β-D-glucopyranoside).

It is also advantageous to choose the active ingredient or the active ingredients from ubiquinones and plastoquinones.

Ubiquinones are characterized by the structural formula 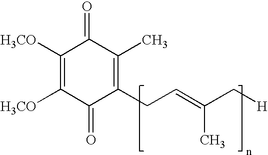
and represent the most widespread and thus the best investigated bioquionones. Depending on the number of isoprene units linked in the side chain, ubiquinones are referred to as Q-1, Q-2, Q-3 etc., or on the number of carbon atoms as U-5, U-10, U-15 etc. They preferably occur with certain chain lengths, e.g. in some microorganisms and yeasts with n=6. In most mammals including man Q10 predominates.

Coenzyme Q10 is particularly advantageous; it is characterized by the following structural formula:

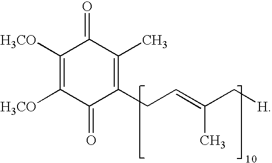

Plastoquinones have the general structural formula

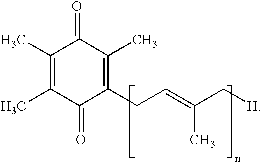

Plastoquinones differ in the number n of isoprene radicals and are indicated accordingly, e.g. PQ-9 (n=9). Furthermore, other plastoquinones having different substituents on the quinone ring exist.

Creatine and/or creatine derivatives are also preferred active ingredients for the purposes of the present invention. Creatine is characterized by the following structure:

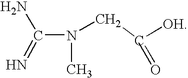

Preferred derivatives are creatine phosphate and creatine sulfate, creatine acetate, creatine ascorbate and the derivatives esterified on the carboxyl group by mono- or polyfunctional alcohols.

A further advantageous active ingredient is L-carnitine [3-hydroxy-4-(trimethylammonio)-butyrobetaine]. Acyl carnitines, which chosen from the group of substances of the following general structural formula 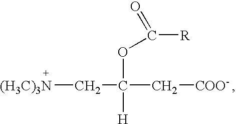
where R is selected from branched and unbranched alkyl radicals with up to 10 carbon atoms are also advantageous active ingredients for the purposes of the present invention. Preference is given to propionylcarnitine and in particular acetylcarnitine. Both enantiomers (D and L form) can be used advantageously for the purposes of the present invention. It may also be advantageous to use any desired enantiomer mixures, for example a racemate of the D and L form.

Further advantageous active ingredients are sericoside, pyridoxol, vitamin K, biotin and aromatic substances.

Furthermore, the active ingredients according to the invention (one or more compounds) can also very advantageously be selected from hydrophilic active ingredients, in particular from the following group:
alpha hydroxy acids, such as lactic acid or salicylic acid, or salts thereof, such as, for example, Na lactate, Ca lactate, TEA lactate, urea, allantoin, serine, sorbitol, glycerol, milk protein, panthenol, chitosan.

The list of the active ingredients or active ingredient combinations mentioned which can be used in the preparations according to the invention should of course not be limiting. The active ingredients can be used individually or in any desired combinations with one another.

The amount of such active ingredients (one or more compounds) in the preparations according to the invention is preferably 0.001 to 30% by weight, particularly preferably 0.05-20% by weight, in particular 1-10% by weight, based on the total weight of the preparation.

The use of the cosmetic or dermatological cleansing emulsions as foam bath, shower bath or tub bath, and as hair shampoo is in accordance with the invention.

The cosmetic or dermatological cleansing emulsions according to the invention are advantageously used as a thickened, in particular cream-like, preparation.

Not least, the use of the cosmetic or dermatological cleansing emulsions for the prophylaxis and/or treatment of inflammatory skin conditions and/or for skin protection in the case of sensitively determined and dry skin is in accordance with the invention.

### DETAILED DESCRIPTION OF THE INVENTION

The examples below, in which washing preparations for hair care and body care are described, are intended to illustrate the compositions according to the invention, but without any restriction of the invention to these examples being intended. The numerical values in the examples are percentages by weight, based on the total weight of the particular preparations.

### FORMULATION EXAMPLES

| | 1 | 2 | 3 | 4 | 5 |
|---|---|---|---|---|---|
| Paraffin oil | 20% | 20% | 20% | 20% | 25% |
| Soybean oil | 20% | 20% | 20% | 20% | 25% |
| Sodium lauryl ether | 11% | 11% | 11% | 11% | 11% |
| sulfate | | | | | |
| Sodium benzoate | 0.3% | — | 0.3% | — | 0.3% |
| Sodium salicylate | 0.2% | — | 0.2% | — | 0.2% |
| Acrylates/C10-C30 alkyl | 1% | 1% | 1% | 1% | 0.8% |
| acrylate crosspolymer | | | | | |
| Talc | 2% | 1% | 2% | 2% | 2% |
| EDTA | 0.5% | — | — | 0.3% | — |
| Iminodisuccinic acid | — | — | 1% | — | — |
| tetrasodium salt | | | | | |
| Sodium hydroxide | 0.2% | 0.2% | 0.2% | 0.2% | 0.2% |
| Phenoxyethanol | — | 0.5% | — | 0.5% | — |
| Parabens | — | 0.2% | — | 0.2% | — |
| Perfume | q.s. | q.s. | q.s. | q.s. | q.s. |
| Water | ad 100 | ad 100 | ad 100 | ad 100 | ad 100 |
| | 6 | 7 | 8 | 9 | 10 |
| Paraffin oil | 40% | 20% | 20% | 20% | 12% |
| Soybean oil | — | 20% | 20% | 20% | 30% |
| Sodium lauryl ether | 11% | 11% | 5% | 15% | 13% |
| sulfate | | | | | |
| Sodium benzoate | 0.3% | — | 0.3% | — | 0.3% |
| Sodium salicylate | 0.2% | — | 0.2% | — | 0.2% |
| Acrylates/C10-C30 alkyl | 1% | 1% | 1% | 1% | 0.8% |
| acrylate crosspolymer | | | | | |
| Talc | 2% | 1% | 0.5% | 0.3% | 5% |
| EDTA | 1% | — | — | 0.3% | — |
| Coenzyme Q10 | — | 0.1% | — | — | — |
| Iminodisuccinic acid | — | — | 1% | 0.3% | — |
| tetrasodium salt | | | | | |
| Sodium hydroxide | 0.2% | 0.2% | 0.2% | 0.2% | 0.2% |
| Phenoxyethanol | — | 0.5% | — | 0.5% | — |
| Parabens | — | 0.2% | — | 0.2% | — |
| Perfume | q.s. | q.s. | q.s. | q.s. | q.s. |
| Water | ad 100 | ad 100 | ad 100 | ad 100 | ad 100 |

1. A cosmetic or dermatological cleansing emulsion comprising:
(a) from 1% to 30% by weight of one or more detergent surfactants which have an HLB value of higher than 15;
(b) from 35% to 50% by weight of one or more oil components;
(c) from 0.1% to 10% by weight of talc;
(d) from 0.2% to 5% by weight of one or more polyacrylates selected from anionic homopolymers and anionic copolymers of at least one of acrylic acid, alkylated acrylic acid and esters thereof; and
(e) from 5% to 60% by weight of water.

2. The emulsion of claim 1, wherein the one or more detergent surfactants have an HLB value of higher than 25.

3. The emulsion of claim 1, wherein the one or more detergent surfactants have an HLB value of higher than 35.

4. The emulsion of claim 1, wherein the one or more detergent surfactants comprise one or more anionic surfactants.

5. The emulsion of claim 4, wherein the one or more anionic surfactants comprise at least one of acylamino acids and salts thereof, sulfonic acids and salts thereof, and sulfuric acid esters.

6. The emulsion of claim 1, wherein the one or more detergent surfactants comprise one or more cationic surfactants.

7. The emulsion of claim 6, wherein the one or more cationic surfactants comprise at least one quaternary surfactant.

8. The emulsion of claim 1, wherein the one or more detergent surfactants comprise one or more amphoteric surfactants.

9. The emulsion of claim 8, wherein the one or more amphoteric surfactants comprise at least one acyl-/dialkylethylenediamine.

10. The emulsion of claim 1, wherein the one or more detergent surfactants comprise one or more nonionic surfactants.

11. The emulsion of claim 10, wherein the one or more nonionic surfactants comprise at least one of an alkanolamide, an ester formed by esterification of a carboxylic acid with at least one of ethylene oxide, glycerol and sorbitan, and an ether.

12. The emulsion of claim 1, wherein the emulsion comprises from 5% to 25% by weight of component (a).

13. The emulsion of claim 2, wherein the emulsion comprises from 10% to 20% by weight of component (a).

14. The emulsion of claim 1, wherein component (a) comprises sodium laureth sulfate.

15. The emulsion of claim 12, wherein component (a) comprises at least one of sodium acyl glutamate, myristoyl sarcosine, TEA lauroyl sarcosinate, sodium lauroyl sarcosinate and sodium cocoyl sarcosinate, sodium/ammonium cocoyl isethionate, dioctyl sodium sulfosuccinate, disodium laureth sulfosuccinate, disodium lauryl sulfosuccinate and disodium undecyleneamido MEA sulfosuccinate, sodium, ammonium, magnesium, MIPA, TIPA laureth sulfate, sodium myreth sulfate and sodium C₁₂₋₁₃ pareth sulfate, sodium, ammonium and TEA lauryl sulfate, benzalkonium chloride, alkylbetaine, alkylamidopropylbetaine and alkylamidopropylhydroxysultaine, sodium acylamphoacetate, disodium acylamphodipropionate, disodium alkylamphodiacetate, sodium acylamphohydroxypropylsulfonate, disodium acylamphodiacetate and sodium acylamphopropionate, cocamide MEA/DEA/MIPA, lauryl glucoside, decyl glycoside and coconut glycoside.

16. The emulsion of claim 1, wherein component (b) comprises at least one of 2-ethylhexyl isostearate, octyldodecanol, isotridecyl isononanoate, isoeicosane, 2-ethylhexyl cocoate, C₁₂₋₁₅ alkyl benzoate, caprylic/capric acid triglyceride, and dicaprylyl ether.

17. The emulsion of claim 2, wherein component (b) comprises at least one of a mixture of C₁₂₋₁₅ alkyl benzoate and 2-ethyl hexyl isostearate, a mixture of C₁₂₋₁₅ alkyl benzoate and isotridecyl isononanoate, and a mixture of C₁₂₋₁₅ alkyl benzoate, 2-ethyl-hexyl isostearate and isotridecyl isononanoate.

18. The emulsion of claim 1, wherein component (c) comprises talc having an average particle size of from 1 μm to 20 μm.

19. The emulsion of claim 18, wherein the emulsion comprises from 0.5% to 5% by weight of component (c).

20. The emulsion of claim 1, wherein the emulsion comprises up to 3% by weight of component (c).

21. The emulsion of claim 20, wherein component (d) comprises a polymer of at least one of acrylic acid and esters thereof, acrylate/alkyl acrylate copolymers and combinations thereof.

22. The emulsion of claim 2, wherein the emulsion comprises from 0.5% to 2% by weight of component (d).

23. The emulsion of claim 13, wherein the emulsion comprises at least one anionic surfactant and at least one C₁₀-C₃₀ alkyl acrylate polymer.

24. The emulsion of claim 1, wherein the emulsion further comprises at least one active ingredient.

25. A cosmetic or dermatological cleansing emulsion comprising:
(a) from 5% to 20% by weight of one or more detergent surfactants which have an HLB value of higher than 15;
(b) from 35% to 50% by weight of one or more oil components;
(c) from 0. 5% to 5% by weight of talc;
(d) from 0.5% to 2% by weight of one or more polyacrylates selected from at least one of anionic homopolymers and anionic copolymers of at least one of acrylic acid, alkylated acrylic acid and esters thereof; and
(e) from 5% to 55% by weight of water.

26. The emulsion of claim 25, wherein the one or more detergent surfactants comprise at least one surfactant having an HLB value of higher than 25.

27. The emulsion of claim 26, wherein the one or more detergent surfactants comprise one or more anionic surfactants.

28. The emulsion of claim 26, wherein the emulsion comprises from 10% to 20% by weight of component (a).

29. The emulsion of claim 28, wherein component (a) comprises sodium laureth sulfate.

30. The emulsion of claim 25, wherein component (a) comprises at least one of sodium acyl glutamate, myristoyl sarcosine, TEA lauroyl sarcosinate, sodium lauroyl sarcosinate and sodium cocoyl sarcosinate, sodium/ammonium cocoyl isethionate, dioctyl sodium sulfosuccinate, disodium laureth sulfosuccinate, disodium lauryl sulfosuccinate and disodium undecyleneamido MEA sulfosuccinate, sodium, ammonium, magnesium, MIPA, TIPA laureth sulfate, sodium myreth sulfate and sodium C₁₂₋₁₃ pareth sulfate, sodium, ammonium and TEA lauryl sulfate, benzalkonium chloride, alkylbetaine, alkylamidopropylbetaine and alkylamidopropylhydroxysultaine, sodium acylamphoacetate, disodium acylamphodipropionate, disodium alkylamphodiacetate, sodium acylamphohydroxypropylsulfonate, disodium acylamphodiacetate and sodium acylamphopropionate, cocamide MEA/DEA/MIPA, lauryl glucoside, decyl glycoside and coconut glycoside.

31. The emulsion of claim 30, wherein component (c) comprises talc having an average particle size of from 1 μm to 20 μm.

32. The emulsion of claim 31, wherein component (d) comprises a polymer of at least one of acrylic acid and esters thereof, acrylate/alkyl acrylate copolymers, and combinations thereof.

33. The emulsion of claim 25, wherein the emulsion comprises from 0.7% to 1.5% by weight of component (d).

34. The emulsion of claim 25, wherein the emulsion comprises at least one anionic surfactant and at least one C₁₀-C₃₀ alkyl acrylate polymer.

35. A foam bath which comprises the emulsion of claim 1.

36. A shower bath which comprises the emulsion of claim 1.

37. A tub bath which comprises the emulsion of claim 1.

38. A hair shampoo which comprises the emulsion of claim 1.

39. A cream-like preparation which comprises the emulsion of claim 1.

40. A method for the treatment or prophylaxis of inflammatory skin conditions, wherein the method comprises applying to at least parts of the skin the emulsion of claim 1.

41. A method of protecting sensitive and dry skin, wherein the method comprises applying to at least parts of the skin the emulsion of claim 1.
